(19) United States
(12) Patent Application Publication
(10) Pub. No.: US 2022/0304244 A1
(43) Pub. Date: Sep. 29, 2022

(54) METHOD FOR PREPARING 13C LABELLED PLANT AND METHOD FOR PREPARING 13C LABELLED BIOCHAR

(71) Applicant: SUZHOU UNIVERSITY OF SCIENCE AND TECHNOLOGY, Suzhou (CN)

(72) Inventors: Yuan LIANG, Suzhou (CN); Xingran LI, Suzhou (CN)

(21) Appl. No.: 17/284,951

(22) PCT Filed: Dec. 16, 2020
(86) PCT No.: PCT/CN2020/136693

§ 371 Date: Apr. 13, 2021

(30) Foreign Application Priority Data
Dec. 17, 2019 (CN) 201911299104.6

(51) Int. Cl. A01G 7/02; C01B 32/05; C01B 32/50; C01B 32/60

(52) U.S. Cl. CPC A01G 7/02; C01B 32/05; C01B 32/50; C01B 32/60; C01P 2006/88

(57) ABSTRACT

The present disclosure discloses a method for preparing a labelled plant and a method for preparing a labelled biochar. The method for preparing a labelled plant includes the following steps: preparing a box; placing a plant that is capable of photosynthesis in the box; preparing a labelled gas in the box, the labelled gas being an isotope-labelled carbon dioxide gas; and leaving the box standing for a preset period of time, during which the plant absorbs the labelled gas to obtain a labelled plant. In the present disclosure, the carbon element in the plant is labelled during the growth of the plant, and the concentration of ¹³CO₂ in the box is controlled by controlling the concentration of Na₂¹³CO₃, where ¹³CO₂ is prepared by chemical methods, which could improve the utilization rate of ¹³C.

### CROSS REFERENCE TO RELATED APPLICATIONS

This application claims the priority of Chinese Patent Application No. 201911299104.6, entitled “methods for preparing labelled plant and labelled biochar” filed with the China National Intellectual Property Administration on Dec. 17, 2019, which is incorporated herein by reference in its entirety.

### TECHNICAL FIELD

The present disclosure relates to the field of biotechnology, and in particular to methods for preparing labelled plants and labelled biochar.

### BACKGROUND ART

At present, most of the pollutants in the soil have poor mobility in the soil and have a long residence time, and some pollutants (such as heavy metals) could not be degraded by microorganisms, which will endanger human health through groundwater and plant root absorption. As a green (i.e. environmentally-friendly) and effective heavy metal chemically stablizing material, biochar could not only be used to fix carbon, improve soil fertility, improve acidity and alkalinity, reduce the loss of nitrogen and phosphorus and other nutrients in soil, but also adsorb pollutants in the soil, which can effectively reduce the mobility of pollutants in the soil, and is an economical and effective soil remediation agent.

However, external conditions such as irrigation, rainfall, and fertilization may have a certain impact on the reaction process and mechanism of the pollutant-biochar-soil system at the solid-liquid interface, thereby affecting the co-migration behavior of pollutants and biochar. Thus, the migrations of biochar and pollutants need to be monitored after biochar is applied to the soil. The migration behavior of biochar in soil could not only affect the stability of biochar itself, but also affect the desorption of pollutants by biochar. Therefore, it is very important to study the migration behavior of biochar in the soil.

The organic carbon in the soil mainly comes from plant residues, so it has a similar composition to that of biochar. Thus, when biochar is applied to the soil, it is difficult to distinguish the organic carbon from the biochar by conventional means.

### SUMMARY

An objective of the present disclosure is to provide a method for preparing a labelled plant to solve the problem of the source of raw materials for isotope-labelled biochar.

The present disclosure provides a method for preparing a labelled plant, comprising the following steps: preparing a box; placing a plant that is capable of photosynthesis in the box; preparing a labelled gas in the box, the labelled gas being an isotope-labelled carbon dioxide gas; and leaving the box standing for a preset period of time, during which the plant absorbs the labelled gas to obtain a labelled plant.

In some embodiments, after preparing the box, the method further comprises disposing a temperature controlling device and a gas mixing device in the box.

In some embodiments, the temperature controlling device is an ice pack or an electronic temperature controlling device; the temperature controlling device is located at the bottom of the box.

In some embodiments, the gas mixing device is an electric fan; the gas mixing device is located at the top of the box.

In some embodiments, the box is made of a transparent material, and the box is sealable.

In some embodiments, the preset period of time is in a range of 6 hours to 12 hours.

In some embodiments, preparing a labelled gas in the box specifically comprises the following steps: placing a container with an acidic solution in the box; and adding an isotope-labelled sodium carbonate solution Na₂¹³CO₃ to the container, to generate a labelled gas in the box.

In some embodiments, the acidic solution is HCl.

Another objective of the present disclosure is to provide a method for preparing a labelled biochar, which can effectively solve the problem that it is difficult to distinguish biochar after applied to the soil from the organic carbon in the soil due to the similar compositions.

The present disclosure provides a method for preparing a labelled biochar, comprising the following steps: preparing a labelled plant according to any one of the above-mentioned methods; pretreating the labelled plant; and pyrolyzing the pretreated labelled plant at a preset temperature.

In some embodiments, pretreating the labelled plant specifically comprises the following steps: inactivating, drying and crushing the labelled plant.

In some embodiments, pyrolyzing the pretreated labelled plant specifically comprises the following steps: placing the pretreated labelled plant in a muffle furnace; introducing a sufficient amount of nitrogen into the muffle furnace; raising the temperature of the muffle furnace to a preset temperature at a constant heating rate; and heating the pretreated labelled plant at the preset temperature for 2 hours to 4 hours.

In some embodiments, the constant heating rate is 15° C./min.

In some embodiments, the preset temperature is between 350° C. and 550° C.

Another objective of the present disclosure is to provide a device for labelling a plant, comprising a box 1 in which a gas mixing device 2, a temperature controlling device 3 and a container 4 are arranged.

In some embodiments, the gas mixing device 2 is an electric fan; the gas mixing device 2 is located at the top of the box 1.

In some embodiments, the temperature controlling device 3 is an ice pack or an electronic temperature controlling device; the temperature controlling device 3 is located at the bottom of the box 1.

The advantages of the present disclosure lie in that: the carbon element in the plant is labelled during the growth of the plant in the present disclosure, and the concentration of ¹³CO₂ in the box is controlled by controlling the concentration of Na₂¹³CO₃, where ¹³CO₂ is prepared by chemical methods, which improves the utilization rate of ¹³C. In addition, the isotope labelling method does not change the physical and chemical properties of the biochar itself, but only increases the abundance of ¹³C, to play a role of labelling and tracking, without adversely affecting the adsorption effect of the biochar on pollutants. The labelled biochar prepared in the present disclosure has a significantly different abundance of ¹³C from those of ordinary biochar and soil. After applied to the soil, the labelled biochar could be tracked by stable isotope technology for the migration route.

### BRIEF DESCRIPTION OF THE DRAWINGS

The specific embodiments of the present disclosure will be described in detail with reference to the following accompanying drawings, to make the technical solutions and other beneficial effects of the present disclosure obvious.

FIG. 1 shows a flowchart illustrating steps of a method for preparing a labelled plant provided by an embodiment of the present disclosure;

FIG. 2 shows a flowchart illustrating steps of step S140 provided by an embodiment of the present disclosure;

FIG. 3 shows a schematic structural diagram of the box provided by an embodiment of the present disclosure;

FIG. 4 shows a flowchart illustrating steps of a method for preparing a labelled biochar provided by an embodiment of the present disclosure;

FIG. 5 shows a flowchart illustrating specific steps of step S440 provided by an embodiment of the present disclosure;

FIG. 6 shows a flowchart illustrating specific steps of step S460 provided by an embodiment of the present disclosure;

FIG. 7 shows a flowchart illustrating specific steps of step S470 provided by an embodiment of the present disclosure.

### DETAILED DESCRIPTION OF THE EMBODIMENTS

The technical solutions in the embodiments of the present disclosure will be clearly and completely described below in conjunction with the drawings in the embodiments of the present disclosure. Obviously, the described embodiments are only a part of the embodiments of the present disclosure, rather than all the embodiments. Based on the embodiments of the present disclosure, all other embodiments obtained by those skilled in the art without creative labor shall fall within the protection scope of the present disclosure.

In the present disclosure, it should be understood that the terms “center”, “longitudinal”, “transverse”, “length”, “width”, “thickness”, “upper”, “lower”, “front”, ” “back”, “left”, “right”, “vertical”, “horizontal”, “top”, “bottom”, “inner”, “outer”, “clockwise”, “counterclockwise” and other directions or positional relationships are based on the directions or positional relationships shown in the drawings, and are only for better describing the present disclosure and simplifying the description, and does not indicate or imply that the pointed device or element must be oriented in a specific orientation, be constructed and operated in a specific orientation. Therefore, it could not be understood as a limitation to the present disclosure. In addition, the terms “first” and “second” are only used for descriptive purposes, and could not be understood as indicating or implying relative importance or implicitly indicating the number of indicated technical features. Therefore, the features defined with “first” and “second” may explicitly or implicitly include one or more features. In the description of the present disclosure, “plurality” means two or more than two, unless specifically defined otherwise.

In the description of the present disclosure, it should be noted that the terms “installation”, “connected” and “connection” should be understood in a broad sense, unless otherwise clearly specified and limited. For example, the terms could refer to a fixed connection, a detachable connection, or a integral connection; they could refer to a mechanical connection, an electrical connection or in a mutual communication; they could refer to a direct connection or an indirect connection through an intermediate medium; they could refer to a communication between the inside of two elements or an interaction relationship between two elements. For those skilled in the art, the specific meanings of the above terms in the present disclosure could be understood according to specific circumstances.

In the present disclosure, unless otherwise clearly specified and defined, the expression that the first feature is “above” or “below” the second feature may includes that the first and second features are in direct contact, or that the first and second features are not in direct contact but through other features between them. Moreover, the expression that the first feature is “above”, “over” and “on” the second feature includes that the first feature is directly above and obliquely above the second feature, or that the level of the first feature is higher than that of the second feature. The expression that the first feature is “below”, “under”, and “beneath” the second feature includes that the first feature is directly below and obliquely below the second feature, or that the level of the first feature is lower than that of the second feature.

The following disclosure provides many different embodiments or examples for realizing different structures of the present disclosure. In order to simplify the disclosure of the present disclosure, the components and settings of specific examples are described below. Of course, they are only examples, and are not intended to limit the disclosure. In addition, the present disclosure may repeat reference numerals and/or reference letters in different examples. Such repetition is for the purpose of simplification and clarity, and does not indicate the relationship between the various embodiments and/or settings discussed. In addition, the present disclosure provides examples of various specific processes and materials, but those skilled in the art may be aware of the application of other processes and/or the use of other materials.

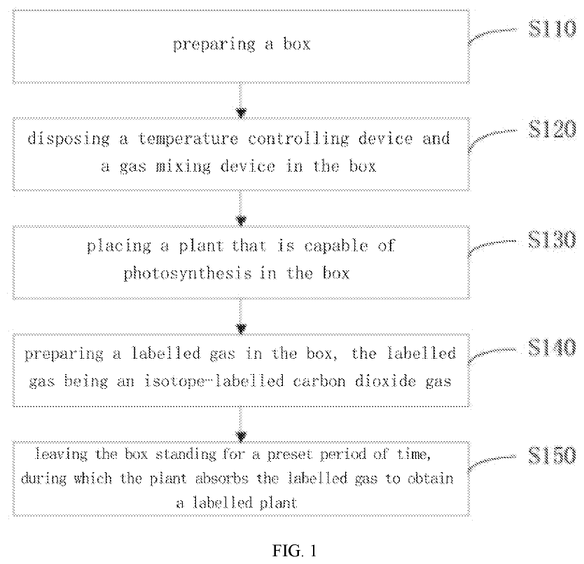

As shown in FIG. 1, a flowchart illustrating steps of the method for preparing a labelled plant provided in this embodiment comprises the following steps S110 to S150:

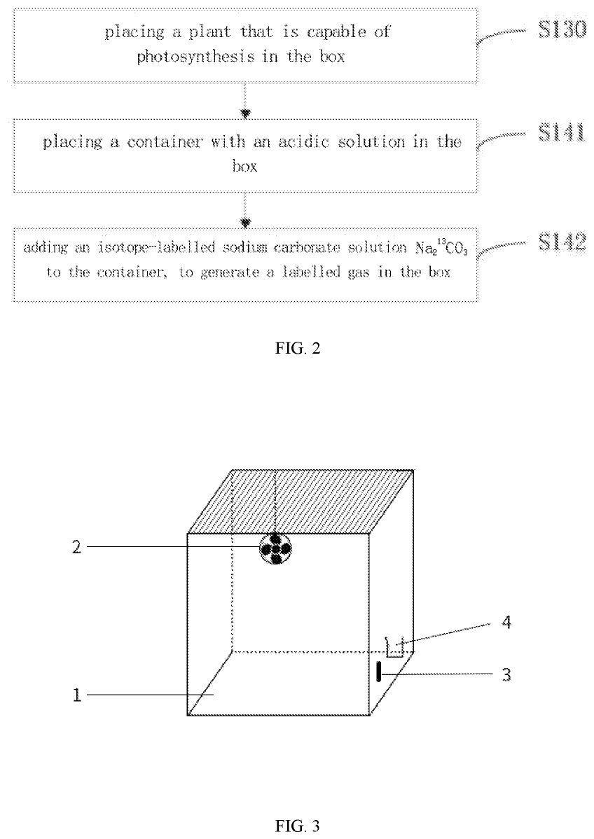

step S110: a box 1 is prepared. In this embodiment, as shown in FIG. 3, the box 1 is a cube in shape, but it is not limited to this, for example, in other embodiments, the box 1 may also be a cuboid in shape; the volume of the box 1 can be determined according to the number of the labelled plant to be planted; the box 1 is made of a transparent material, for example, transparent glass; the box 1 is sealable, so that the gas in the box 1 does not leak out, which improves the utilization rate of the gas in the box 1 and reduces the cost;

step S120: a temperature controlling device 3 and a gas mixing device 2 are disposed in the box 1. In this embodiment, the temperature controlling device 3 is used to adjust the temperature in the box 1 so that the temperature in the box 1 is within the optimal temperature range for photosynthesis; the temperature controlling device 3 is generally disposed at the bottom of the box 1, compared with other positions, the temperature controlling device 3 is closer to the plant in the box 1, which makes the temperature gradient range in the area where the plant is located is closer to the actual temperature adjusted by the temperature controlling device 3, which controls the temperature of the box 1 to prevent decreased photosynthesis efficiency of the plant due to high temperature; the temperature controlling device 3 may be an ice pack or an electronic temperature controlling device; the gas mixing device 2 is used for evenly distributing the gas (mainly labelled gas) in the box 1, so that each part (for example, leaves, stems, roots) of each plant in the box 1 is exposed to the labelled gas with a same concentration; the gas mixing device 2 is generally arranged at the top of the box 1, and the gas mixing device 2 may be an electric fan;

step S130: a plant that is capable of photosynthesis is placed in the box 1.

In this embodiment, the plant is corn, but it is not limited thereto. In other embodiments, the plant may also be cotton, etc.

Refer to FIG. 2. Step S140: a labelled gas in the box 1 is prepared, where the labelled gas is an isotope-labelled carbon dioxide gas. In this embodiment, the isotope-labelled carbon dioxide gas is ¹³CO_{2,} and step S140 of preparing the labelled gas in the box 1 specifically comprises the following steps:

step S141: a container 4 with an acidic solution is placed in the box 1;

step S142: an isotope-labelled sodium carbonate solution Na₂¹³CO₃ is added to the container 4 to generate the labelled gas in the box 1.

In step S141 and step S142, the capacity of the container 4 is determined by the number of the labelled plant to be planted, and the acidic solution is HCl (hydrochloric acid). In actual operation, the molar amount of HCl in the container 4 should be sufficient, so the concentration of ¹³CO₂ in the box 1 could be controlled if the amount of Na₂¹³CO₃ is controlled; the chemical reaction formula to generate ¹³CO₂ is Na₂¹³CO₃+HCl→NaCl+H₂O+¹³CO₂, but not limited to this.

Step S150: the box is left standing for a preset period of time, during which the plant absorbs the labelled gas to obtain a labelled plant. In this embodiment, the preset period of time is in a range of 6 hours to 12 hours. Since photosynthesis is carried out in the box 1, the optimal preset period of time for photosynthesis is estimated according to the concentration of carbon dioxide and the number of the plant in the box; when the preset period of time is shorter, the carbon dioxide labelling efficiency is lower, and when the preset period of time is longer, the temperature in the box will increase, which will damage the growth of the plant. In addition, it should be noted that in order to increase the abundance of ¹³C in the plant, in actual operation, the above steps S110 to S150 need to be repeated multiple times, with an interval of one week, and the number of repetitions is determined according to the actual growth condition of the plant, but at least five repetitions could make the abundance of ¹³C in the plant distinguished from that of the ordinary plant. If the plant growth is too tall to be completely put into the box 1, the labelling could be ended when five or more repetitions are completed.

The advantage of the present disclosure is that in the present disclosure, the carbon element in the plant is labelled during the growth of the plant, and the concentration of ¹³CO₂ in the box is controlled by controlling the concentration of Na₂¹³CO₃, where ¹³CO₂ is prepared by chemical methods, which could improve the utilization rate of ¹³C.

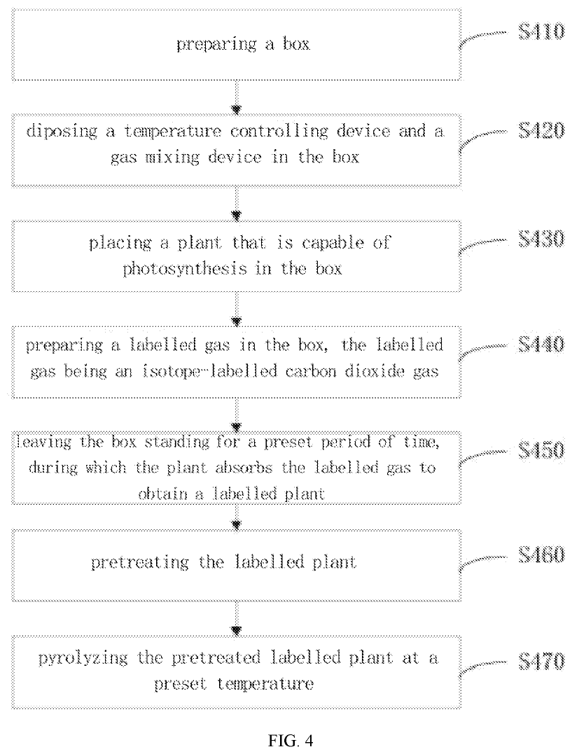

As shown in FIG. 4, it shows a flowchart illustrating steps of a method for preparing a labelled biochar provided by an embodiment of the present disclosure, and the method comprises the following steps S410 to S470:

step S410: a box 1 is prepared. In this embodiment, the box 1 is a cube in shape, but it is not limited to this, for example, in other embodiments, the box 1 may also be a cuboid in shape; the volume of the box 1 could be determined according to the number of labelled plant to be planted; the box 1 is made of a transparent material, for example, transparent glass; the box 1 is sealable, so that the gas in the box 1 does not leak out, which improves the utilization rate of the gas in the box 1 and reduces the cost;

In step S420, a temperature controlling device 3 and a gas mixing device 2 are disposed in the box 1, as shown in FIG. 3. In this embodiment, the temperature controlling device 3 is used to adjust the temperature in the box 1 so that the temperature in the box 1 is within the optimal temperature range for photosynthesis; the temperature controlling device 3 is generally disposed at the bottom of the box 1, compared with other positions, the temperature controlling device 3 is closer to the plant in the box 1, which makes the temperature gradient range in the area where the plant is located is closer to the actual temperature adjusted by the temperature controlling device 3; the temperature of the box 1 is controlled to prevent decreased photosynthesis efficiency of the plant due to high temperature; the temperature controlling device 3 may be an ice pack or an electronic temperature controlling device; the gas mixing device 2 is used for evenly distributing the gas (mainly labelled gas) in the box 1, so that each part (for example, leaves, stems, roots) of each plant in the box 1 is exposed to the labelled gas with a same concentration; the gas mixing device 2 is generally arranged at the top of the box 1, and the gas mixing device 2 may be an electric fan;

step S430: a plant that is capable of photosynthesis is placed in the box 1.

In this embodiment, the plant is corn, but it is not limited thereto. In other embodiments, the plant may also be cotton, etc.

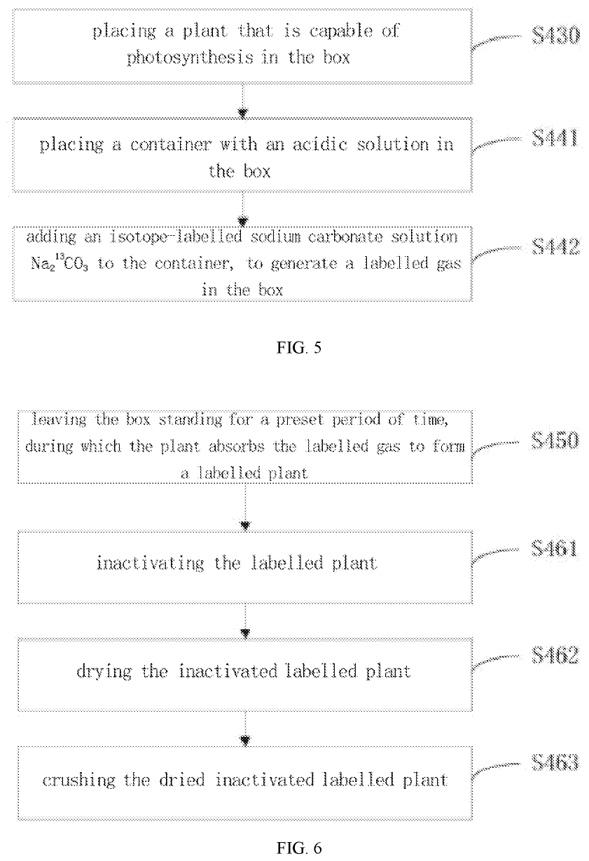

As shown in FIG. 5, in step S440, a labelled gas is prepared in the box 1, wherein the labelled gas is an isotope-labelled carbon dioxide gas. In this embodiment, the isotope-labelled carbon dioxide gas is ¹³CO₂, and step S140 of preparing the labelled gas in the box 1 specifically comprises the following steps:

step S441: a container 4 with an acidic solution is placed in the box 1;

step S442: an isotope-labelled sodium carbonate solution Na₂¹³CO₃ is added to the container 4 to generate the labelled gas in the box 1.

In step S441 and step S442, the capacity of the container 4 is determined by the number of the labelled plant to be planted, and the acidic solution is HCl (hydrochloric acid). In actual operation, the molar amount of HCl in the container 4 should be sufficient, so the concentration of ¹³CO₂ in the box 1 could be controlled if the amount of Na₂¹³CO₃ is controlled; the chemical reaction formula to generate ¹³CO₂ is Na₂¹³CO₃+HCl→NaCl+H₂O+¹³CO₂, but not limited to this.

Step S450: the box is left standing for a preset period of time, during which the plant absorbs the labelled gas to obtain a labelled plant.

In this embodiment, the preset period of time is in a range of 6 hours to 12 hours. Since photosynthesis is carried out in the box 1, the optimal preset period of time range for photosynthesis is estimated according to the concentration of carbon dioxide and the number of the plant in the box; when the preset period of time is shorter, carbon dioxide labelling efficiency is lower, and when the preset period of time is longer, the temperature in the box will increase, which will damage the growth of the plant. In addition, it should be noted that in order to increase the abundance of ¹³C in the plant, in actual operation, the above steps S410 to S450 need to be repeated multiple times, with an interval of one week, and the number of repetitions is determined according to the actual growth condition of the plant, but at least five repetitions could make the abundance of ¹³C in the plant distinguished from that of the ordinary plant. If the plant growth is too tall to be completely put into the box 1, the labelling could be ended when five or more labelling is completed.

As shown in FIG. 6, step S460 of pretreating the labelled plant, specifically comprises the steps:

step S461: the labelled plant is inactivated.

step S462: the inactivated labelled plant is dried.

step S463: the dried inactivated labelled plant is crushed.

In step S461, step S462, and step S463, in actual operation, it is necessary to compare the abundance of ¹³C in the labelled plant and the unlabelled plant. Therefore, one labelled plant and one naturally-growing unlabelled plant are selected, inactivated, and dried; then the leaves, stems and roots of the dried corn are crushed separately.

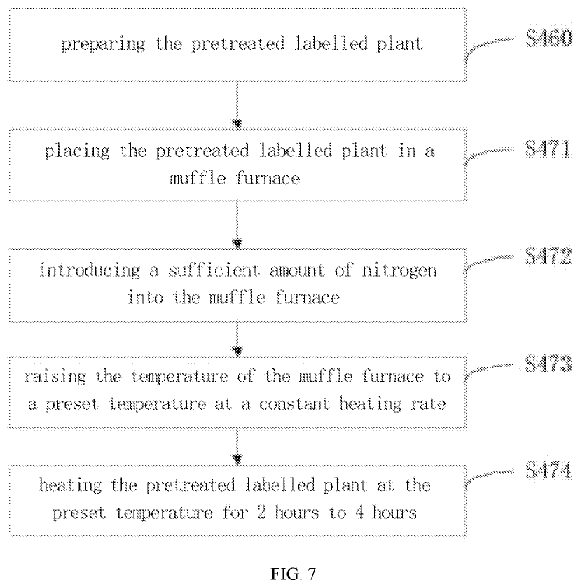

As shown in FIG. 7, in step S470, the pretreated labelled plant is pyrolyzed at a preset temperature. In this embodiment, pyrolyzing the pretreated labelled plant at a preset temperature specifically comprises the steps:

step S471: the labelled plant is placed in a muffle furnace;

step S472: a sufficient amount of nitrogen is passed into the muffle furnace;

step S473: the temperature of the muffle furnace is raised to the preset temperature at a constant heating rate;

step S474: the labelled plant is heated at the preset temperature for 2 hours to 4 hours.

In step S471, step S472, step S473, and step S474, the constant heating rate is 15° C./min, and the preset temperature is between 350° C. and 550° C. In actual operation, the crushed labelled plant is placed in the muffle furnace, a sufficient amount of nitrogen is introduced to limit the oxygen content, and the labelled plant is heated at 550° C. for 3 hours in the muffle furnace.

The results are shown in Table 1 and Table 2. In addition, it should be noted that after the heating is completed, the labelled biochar is placed under natural conditions to cool to room temperature, and finally the prepared labelled biochar is sieved and stored for later use. In this embodiment, the abundance of ¹³C is measured by the MAT 253Plus isotope mass spectrometer, with conditions of hydrochloric acid with a concentration of 0.1 mol/L, sodium carbonate with a concentration of 0.5 mol/L, and a sufficient amount of hydrochloric acid. Table 1 is a statistical table of the abundance of ¹³C in different parts of the labelled plant and the natural plant, and Table 2 is a statistical table of the abundance of ¹³C in biochar and background soil.

TABLE 1
Statistical results of the abundance of ¹³C in different
parts of the labelled plant and natural plant

| Sample | Leaf | Leaf | Leaf | Stem | Stem | Stem | Root | Root |
|---|---|---|---|---|---|---|---|---|
| Part | \textcircled{1} | \textcircled{2} | \textcircled{3} | \textcircled{1} | \textcircled{2} | \textcircled{3} | \textcircled{1} | \textcircled{2} |
| Labelled plant | 225.9 | 224.7 | 221.0 | 284.2 | 270.8 | 274.9 | 322.2 | 382.4 |
| (the abundance | | | | | | | | |
| of ¹³C) | | | | | | | | |
| Natural plant | 76.5 | 85.9 | 83.4 | 65.9 | 67.1 | 70.0 | 51.8 | 52.7 |
| (the abundance | | | | | | | | |
| of ¹³C ) | | | | | | | | |

TABLE 2
Statistical results of the abundance of ¹³C in biochar and background soil

| | Labelled | Natural | Background |
|---|---|---|---|
| Type | biochar | biochar | soil |
| The abundance of ¹³C | 249.25 | −25.65 | −23.47 |

It is found that the abundance of ¹³C in each part of the labelled plant obtained by the preparation method of labelled biochar is significantly higher than that of the natural plant. For example, the abundance of ¹³C in leaf \textcircled{1} of the labelled plant in Table 1 is significantly higher than that of the natural plant. It can be seen that the carbon element in the plant is labelled during the growth of the plant, and the concentration of ¹³CO₂ in the box is controlled by controlling the concentration of Na₂¹³CO₃, which can effectively increase the abundance of ¹³C in the plant. The natural biochar in Table 2 has a abundance of ¹³C close to that of the background soil, so the labelled biochar has better traceability.

The advantage of the present disclosure is that in the present disclosure, the carbon element in the plant is labelled during the growth of the plant, and the concentration of ¹³CO₂ in the box is controlled by controlling the concentration of Na₂¹³CO_{3, where} ¹³CO₂ is prepared by chemical methods, which could improve the utilization rate of ¹³C. In addition, the isotope labelling method does not change the physical and chemical properties of the biochar itself, but only increases the abundance of ¹³C, to play a role of labelling and tracking, without adversely affecting the adsorption effect of the biochar on pollutants. The labelled biochar prepared by the present disclosure has a significantly different abundance of ¹³C from those of ordinary biochar and soil. After applied to the soil, the labelled biochar could be tracked by stable isotope technology for the migration route.

Specific examples are used to illustrate the principles and implementation of the present disclosure. The descriptions of the above embodiments are only used to help understand the technical solutions and core ideas of the present disclosure; those skilled in the art should understand that: the technical solutions recorded in the foregoing embodiments could be modified, or some of the technical features could be equivalently replaced; these modifications or replacements do not cause the essence of the corresponding technical solutions to deviate from the scope of the technical solutions of the embodiments of the present disclosure.

1. A method for preparing a labelled plant, comprising:
preparing a box;
placing a plant that is capable of photosynthesis in the box;
preparing a labelled gas in the box, the labelled gas being an isotope-labelled carbon dioxide gas; and
leaving the box standing for a preset period of time, during which the plant absorbs the labelled gas to obtain a labelled plant.

2. The method for preparing a labelled plant as claimed in claim 1, further comprising:
after preparing the box, disposing a temperature controlling device and a gas mixing device in the box.

3. The method for preparing a labelled plant as claimed in claim 2, wherein the temperature controlling device is an ice pack or an electronic temperature controlling device; the temperature controlling device is located at the bottom of the box.

4. The method for preparing a labelled plant as claimed in claim 2, wherein the gas mixing device is an electric fan; the gas mixing device is located at the top of the box.

5. The method for preparing a labelled plant as claimed in claim 1, wherein the box is made of a transparent material, and the box is sealable.

6. The method for preparing a labelled plant as claimed in claim 1, wherein the preset period of time is in a range of 6 hours to 12 hours.

7. The method for preparing a labelled plant as claimed in claim 1, wherein preparing a labelled gas in the box specifically comprises:
placing a container with an acidic solution in the box; and
adding an isotope-labelled sodium carbonate solution Na₂¹³CO₃ to the container, to generate a labelled gas in the box.

8. The method for preparing a labelled plant as claimed in claim 7, wherein the acidic solution is HCl.

9. A method for preparing a labelled biochar, comprising:
preparing a labelled plant according to the method for preparing the labelled plant as claimed in claim 1;
pretreating the labelled plant; and
pyrolyzing the pretreated labelled plant at a preset temperature.

10. The method for preparing a labelled biochar as claimed in claim 9, wherein pretreating the labelled plant specifically comprises:
inactivating the labelled plant;
drying the inactivated labelled plant; and
crushing the dried inactivated labelled plant.

11. The method for preparing a labelled biochar as claimed in claim 9, wherein pyrolyzing the pretreated labelled plant specifically comprises:
placing the pretreated labelled plant in a muffle furnace;
introducing a sufficient amount of nitrogen into the muffle furnace;
raising the temperature of the muffle furnace to a preset temperature at a constant heating rate; and
heating the pretreated labelled plant at the preset temperature for 2 hours to 4 hours.

12. The method for preparing a labelled biochar as claimed in claim 11, wherein the constant heating rate is 15° C./min.

13. The method for preparing a labelled biochar as claimed in claim 11, wherein the preset temperature is between 350° C. and 550° C.

14. A device for labelling a plant, comprising a box in which a gas mixing device, a temperature controlling device and a container are arranged.

15. The device as claimed in claim 14, wherein the gas mixing device is an electric fan; the gas mixing device is located at the top of the box.

16. The device as claimed in claim 14, wherein the temperature controlling device is an ice pack or an electronic temperature controlling device; the temperature controlling device is located at the bottom of the box.
